(19) United States
(12) Patent Application Publication
(10) Pub. No.: US 2016/0206181 A1
(43) Pub. Date: Jul. 21, 2016

(54) ENDOSCOPE SHAFT

(71) Applicant: OLYMPUS WINTER & IBE GMBH, Hamburg (DE)

(72) Inventor: Gregor KIEDROWSKI, Hamburg (DE)

(73) Assignee: OLYMPUS WINTER & IBE GmbH, Hamburg (DE)

(21) Appl. No.: 14/914,158

(22) PCT Filed: Sep. 24, 2014
(86) PCT No.: PCT/EP2014/002582

§ 371 Date: Feb. 24, 2016

(30) Foreign Application Priority Data
Oct. 2, 2013 (DE) 10 2013 016.302.1

(51) Int. Cl. A61B 1/00

(52) U.S. Cl. CPC A61B 1/00128; A61B 1/00105

(57) ABSTRACT

A shaft of an endoscope including a locking coupling which is designed to connect the end piece of the shaft to a counter piece, and which includes a coupling piece which is mounted on the end piece or on the counter piece so that it can be slidingly moved transversely to the axis thereof, the coupling piece being designed so it can be placed, when being moved in a traverse manner, in or out of the locking engagement with the counter piece or end piece, the transverse bearing of the coupling piece is provided with a locking device which can be brought into contact for suppressing the transverse movement.

The invention relates to a shaft for an endoscope according to the preamble of claim 1.

Such a shaft is disclosed in U.S. Pat. No. 5,151,101 A. The locking coupling enables a precise quick connection of the shaft with a counter piece. The counter piece may, for example, as shown in said publication, be the coupling cone of the main body of the endoscope. This coupling may also be used, for example, for the coupling of an outer shaft to an inner shaft.

In prototypical couplings, a spring is provided which biases the coupling piece in its transverse movement in the direction of engagement. A secure hold is thus ensured.

The spring should not be made too strongly, as otherwise the spring force to be overcome would be too great and the operation would be generally difficult. If, however, a spring force is selected which is too low, the danger exists that the locking engagement disengages as a result of unintentional touching of the coupling piece or vibration. If this vibration occurs during an operation, this can be unpleasant for all involved.

The object of the present invention is to improve the security of the locking engagement of a prototypical shaft of an endoscope with easy operability.

This object is achieved by the features of the characterizing part of claim 1.

According to the invention, an engageable locking device is provided for suppressing transverse movement. This may be switched on and off, so that the coupling piece is locked or unlocked. In the locked state, transverse movement is prevented. A locking with the present locking engagement thus secures this completely, so that all instability can be avoided. A very weak spring can be selected, which is easy to operate.

The coupling piece can be moved with a transverse movement with respect to the longitudinal axis of one of the pieces to be connected into a position which releases the locking engagement or which engages the locking engagement. By means of the locking device, the transverse movement of the coupling piece into the releasing position, which is necessary for unlocking or releasing the locking engagement between the end piece of the shaft and the counter piece, can be suppressed or blocked. For this purpose, it is contemplated that the locking device can be selectively manipulated into a blocking position or into a release position, preferably by rotation of a handle arranged on the coupling piece. In particular, it is provided that any transverse movement of the coupling piece can be suppressed or blocked with the securing device.

The coupling piece may be mounted on the counter piece or on the end piece and lockingly engage in the respective other piece. According to claim 2, the mounting advantageously takes place on the end piece, as is also shown in the aforementioned publication.

According to claim 3, the coupling piece is advantageously mounted for its transverse movement with a socket on a pin of the end piece, in the manner shown in the aforementioned publication. Here, the socket is used in a rotatable configuration as a locking device, which yields structural advantages.

According to claim 4, the coupling piece may alternatively be mounted on a second pin which has a switching member which can be brought into locking engagement. This, too, is a simple and effective solution to the locking problem.

According to claim 5, the coupling piece advantageously surrounds the end piece in the form of a ring, which is also an already-proven structure from the aforementioned publication.

In the drawings, the invention is shown exemplarily and schematically.

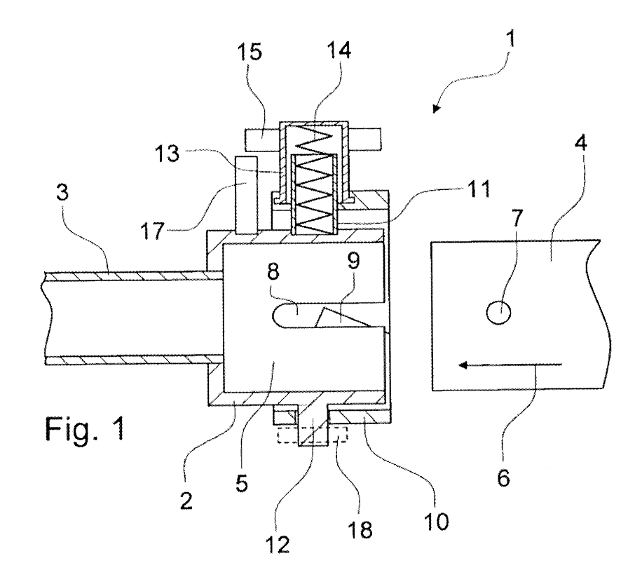

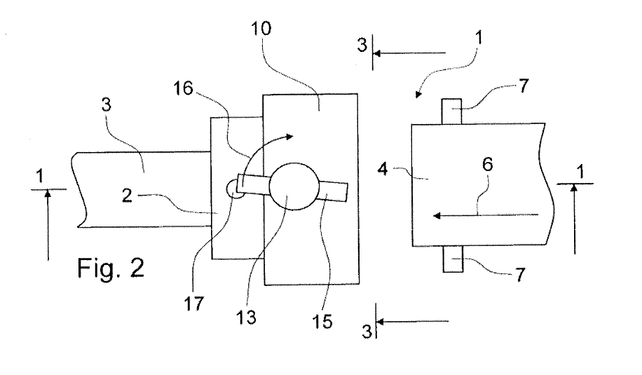

FIG. 1 shows an axial section through an inventive locking coupling, taken along line 1-1 in FIG. 2,

FIG. 2 shows a plan view of the locking coupling of FIG. 1 and

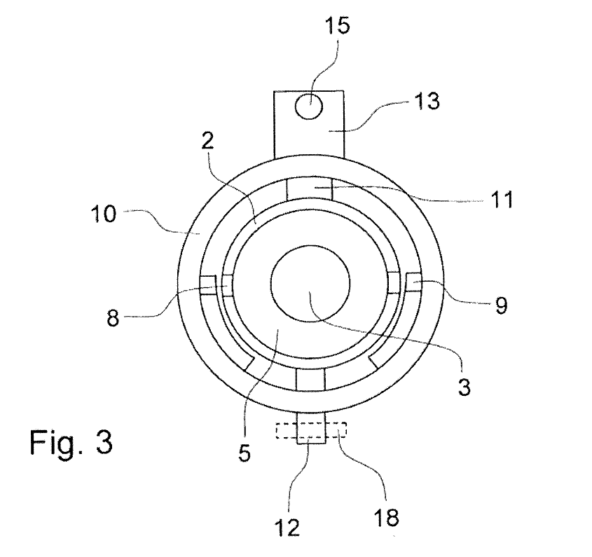

FIG. 3 shows a section along line 3-3 in FIG. 2.

FIGS. 1 to 3 shown different views of a locking coupling 1, with which an end piece 2, which is attached to the proximal end of a hollow shaft 3, can be coupled with a counter piece 4. The counter piece is, for example, part of the handle or of the main body of an endoscope, not shown otherwise, such as a resectoscope. The shaft 3 is intended to be removable, for example for exchanging for a shaft of another type or for cleaning purposes. The coupling should therefore be easy and quick to operate.

The end piece 2 encloses a cavity 5, which is open toward the counter piece 4 and which has an inner cross-section corresponding to the outer cross-section of the counter piece 4, which can thus be mounted in the end piece 2 in a manner fitting free from play after insertion in the direction of the arrow 6.

The counter piece 4 may, for example, also be hollow, in order to enable passage into the shaft 3, or it may support instruments which are advanced into the shaft 3 during coupling.

The counter piece 4 has projecting coupling pins 7 on opposite sides, which coupling pins 7 engage in slots 8 in the side wall of the end piece 2 upon advancement in the direction of the arrow 6, said slots 8 being arranged in parallel to the axis of the shaft 3 and open toward the proximal end. The coupling pins 7 protrude laterally beyond the slots 8 past the outer surface of the end piece 2 and arrive at engagement with locking catches 9, which are arranged on both sides outside the slots 8.

The locking catches 9 are mounted on the inside of a coupling piece 10 which annularly encircles the end piece 2, as is evident from FIG. 1.

FIG. 3 shows a view of the end piece 2 in the axial direction, seen in the direction of insertion of the arrow 6. The slots 8 and the locking latches 9 can be seen, as well as the annular formation of the coupling piece 10 about the end piece 2.

As shown in FIG. 3, in the illustrated embodiment the cross-section of the locking coupling 1 is round. Thus, the end piece 2 and the coupling piece 10 have round cross-sections. Likewise and fitting thereto, the counter piece 4 also has a round cross-section, in order to fit the cavity 5 of the end piece 2.

However, other cross-sections, such as oval or rectangular, are also possible. The coupling piece 10 need not necessarily be formed as a closed ring. It may also be formed, for example, as a interrupted ring with gaps.

A further possibility for variation is the fit of the counter piece 4 in the end piece 2. In the illustrated embodiment, the cavity 5 has parallel inner walls. However, these may also be conical. In this case, the counter piece 4 is would also be provided with conical outer walls. The result would be a structure similar to that of the aforementioned publication.

As shown in FIGS. 1 and 3, the coupling piece 10 is mounted on the end piece 2 in the transverse direction, i.e. transversely to the axis of the shaft 3. For this purpose, a first pin 11 and a second pin 12 are opposingly mounted on the end piece 2. A socket 13 which is displaceable in the direction of the two pins is mounted on the first pin 11. As shown in FIG. 1, the socket 13 is mounted on the coupling piece 10 in a manner which is fixed in the transverse direction, but rotatable about the common axis of the two pins 11 and 12.

If the surgeon presses with his finger against the socket 13, which is closed at its free end, he thus presses the coupling piece 10 in the direction of the axis of the two pins 11 and 12 against the force of a spring 14, which is arranged in the hollow first pin 11 and is supported on one side on the coupling piece 10 and on the other side against the closed outer end of the socket 13.

In the illustrated embodiment, the socket 13 is provided with a T-handle 15. This can be engaged in order to turn the socket 13, as is indicated in FIG. 2 by the arrow 16.

The T-handle 15 also serves as a stop against a support 17, which is mounted on the end piece 2.

The second pin 12 is guided in a bore of the coupling piece 10, so that a double-sided, clean guiding of the coupling piece 10 results in the transverse direction, i.e. in the direction of the axis of the two pins 11 and 12.

As already mentioned, the locking catches 9 are fixed on the inner side of the coupling piece 10 and thus move therewith. If the counter piece 4 is pressed into the cavity 5 of the end piece 2 in the direction of the arrow 6, the coupling pins 7 move into the slots 8 and arrive against the locking catches 9, which can move away together with the coupling piece 10 in the direction of the second pin 12. The spring 14 is thus compressed. If the coupling pins 7 overcome the two locking catches 9, they can spring back under the force of the spring 14 and then secure the coupling pins 7 in the end regions of the slots 8. the locking engagement is thus secured. For releasing, the coupling piece 10 with the locking catches 9 is pushed in the direction of the second pin 12 through pressure on the top of the socket 13, so that the coupling pins 7 come free and the counter piece 4 can be withdrawn.

Alternatively to the illustrated construction, the locking engagement may be designed in a different way. For example, the coupling piece 10 may be slidably mounted on the counter piece 4. The coupling pins 7 could then be mounted internally on the coupling piece 10. The locking catches 9 would thereby be mounted externally on the end piece 2.

In case of accidental contact with the socket 13 or the coupling piece 10, or in case of shock load, unintentional release of the latching may result, in particular with weak springs which ease operation.

To prevent this, the latching can be disabled. For this purpose, the socket 13 is brought with the T-handle 15 into the position of FIG. 2. The T-handle 15 is then in alignment with the support 17 and immediately thereabove, as is shown in FIG. 1. If an attempt is now made to disengage the coupling, i.e. to move the coupling piece 10 in the direction of the 2nd pin 12, the T-handle 15 abuts against the support 17 and prevents this. The coupling is locked in a latched position.

To release the locking, the T-handle 15 is moved in the direction of the arrow 16, as is shown in FIG. 2. The T-handle 15 then comes free from the support 17 and the coupling piece 10 can be moved freely in order to disengage.

An alternative embodiment of the latching locking device is additionally shown in FIG. 1. A switching member 18, only shown schematically with a dashed line, is arranged on the second pin 12, said switching member 18 being switchable between a locking and a non-locking position.

In the illustrated position, the switching member 18 protrudes laterally beyond the second pin 12 and prevents the motion of the coupling piece 10 with respect to the end piece 2 which is necessary for unlocking. In a manner not shown, the switching member 18 is switched such that it is completely removed or no longer projects laterally, so that the coupling piece 10 is freely movable again.

### LIST OF REFERENCE CHARACTERS

1 locking coupling
2 end piece
3 shaft
4 counter piece
5 cavity
6 arrow
7 coupling pin
8 slot
9 locking catch
10 coupling piece
11 first pin
12 second pin
13 socket
14 spring
15 T-handle
16 arrow
17 support
18 switching member

1. A shaft of an endoscope comprising a locking coupling which is designed to connect an end piece of the shaft to a counter piece, and which comprises a coupling piece which is mounted on the end piece or on the counter piece so that it can be slidingly moved transversely to the axis thereof, said coupling piece being designed so it can be placed, when moved in a traverse manner, in or out of the locking engagement with the counter piece or end piece and such that it is supported in the direction of locking engagement by means of a spring, wherein the transverse bearing of the coupling piece is provided with a locking device which can be brought into contact for suppressing the transverse movement for releasing the locking engagement.

2. The shaft according to claim 1, wherein the coupling piece is mounted on the end piece.

3. The shaft according to claim 1, wherein, for its transverse movement, the coupling piece is mounted with a socket on a first pin fixed on the end piece parallel to the transverse movement, wherein the socket is mounted on the coupling piece such that it may rotate but is longitudinally fixed with respect to the axis of the first pin and wherein the socket is designed such that in one rotating position it is locking against the traverse movement.

4. The shaft according to claim 1, wherein, for its transverse movement, the coupling piece is mounted with a transverse bore on a second pin fixed parallel to the transverse movement, wherein a switching member is mounted on the second pin which can be brought in an engagement with the coupling piece such that it is locking the transverse movement.

5. The shaft according to claim 1, wherein the coupling piece annularly surround the end piece.

6. The shaft according to claim 2, wherein, for its transverse movement, the coupling piece is mounted with a socket on a first pin fixed on the end piece parallel to the transverse movement, wherein the socket is mounted on the coupling piece such that it may rotate but is longitudinally fixed with respect to the axis of the first pin and wherein the socket is designed such that in one rotating position it is locking against the traverse movement.

7. The shaft according to claim 2, wherein, for its transverse movement, the coupling piece is mounted with a transverse bore on a second pin fixed parallel to the transverse movement, wherein a switching member is mounted on the second pin which can be brought in an engagement with the coupling piece such that it is locking the transverse movement.

8. The shaft according to claim 2, wherein the coupling piece annularly surround the end piece.

9. The shaft according to claim 3, wherein the coupling piece annularly surround the end piece.

10. The shaft according to claim 4, wherein the coupling piece annularly surround the end piece.

11. The shaft according to claim 6, wherein the coupling piece annularly surround the end piece.

12. The shaft according to claim 7, wherein the coupling piece annularly surround the end piece.
